(19) United States
(12) Patent Application Publication
(10) Pub. No.: US 2006/0024236 A1
(43) Pub. Date: Feb. 2, 2006

(54) Bowel preparation for virtual colonoscopy

(76) Inventor: Perry J. Pickhardt, Madison, WI (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 10/898,954

(22) Filed: Jul. 27, 2004

(51) Int. Cl. A61K 49/04; A61K 33/42; A61K 33/04

(52) U.S. Cl. 424/9.45; 424/709; 424/601

(57) ABSTRACT

The instant invention combines three distinct components to form a single effective bowel preparation for virtual colonoscopy (VC) examination: 4) a cathartic for colonic cleansing; 5) barium sulfate for stool tagging; and 6) water soluble iodinated contrast for colonic fluid opacification. Each component has been employed previously for VC preparation with sub-optimal results. The unique bowel preparation described herein combines these components and has been proven highly effective in a large, prospective multi-center VC screening trial. In addition to being highly efficient, the packaging of these three components into a single preparation kit is much more convenient for patients and their referring physicians. The specific timing and dosage of each component can vary within an acceptable effective range.

### BACKGROUND OF THE INVENTION

Colorectal cancer (CRC) represents a major health care problem throughout the developed world. In the U.S. alone, approximately 150,000 new cases of CRC are diagnosed every year resulting in approximately 55,000 deaths, making it the second-leading cause of cancer death (See: Jemal A., et al. Cancer Statistics, 2003. CA Cancer J Clin 2003; 53:5-26). Risk factors for the development of colon cancer include a low fiber diet, a family history of colon cancer, inflammatory bowel disease, multiple polyps (hereditary and non-hereditary), and a previous history of colon cancer or adenomatous polyps. The incidence of CRC increases with age, especially for individuals over 50 years old. It should be pointed out that the majority of all CRC cases (approximately 75%) occur in patients without any risk factors for developing cancer.

CRC arises from small lesions called polyps. Because polyps grow slowly, and when removed early in their growth can prevent progression to invasive cancer, CRC is largely preventable by effective screening for pre-cancerous polyps and screening for CRC is widely accepted. Current screening options available for CRC include digital rectal examination, fecal occult blood testing, sigmoidoscopy, barium enema, and conventional (fiberoptic) colonoscopy (CC). Unfortunately, only about half of all adults over the age of 50 years have been screened by any means, and only a small fraction have been evaluated by CC (See: Colorectal Cancer Test Use Among Persons Aged >or =50 Years—United States, 2001. MMWR Morb Mortal Wkly Rep 2003; 52:193-196). Despite the availability of these screening examinations, the incidence of CRC remains high because of a reluctance of patients to undergo the screening procedure(s). More widespread detection of the disease should eventually reduce the mortality from the disease. If greater participation in effective colorectal screening were to occur, many additional lives would be saved.

Virtual colonoscopy (VC) represents a very promising screening tool for CRC that could significantly increase the number of adults being screened. Virtual colonoscopy is a test that combines a Computer Assisted Tomography (CT) scanner, sophisticated image processing computers, and skilled radiologists to actually recreate and evaluate the inner surface of the colon. The CT scanner provides the x-ray images and the image processing computers create 2-D and 3-D displays for the final interpretation. Rapid advances in VC technique now allow the potential for effective non-invasive screening. Although the initial VC trials in low-prevalence populations were disappointing (See: Johnson C. D., et al. Prospective Blinded Evaluation of Computed Tomographic Colonography for Screen Detection of Colorectal Polyps. Gastroenterology 2003; 125:311-319; and Cotton P. B., et al. Virtual Colonoscopy: Final Results from a Multi-Center Study. Gastro Endo 2003; 57:AB 174), a prospective multi-center screening trial recently published as the lead article in the New England Journal of Medicine establishes VC as an effective screening tool that is comparable to CC (See: Pickhardt P. J, et al. CT Virtual Colonoscopy to Screen for Colorectal Neoplasia in Asymptomatic Adults. N Engl J Med 2003; 349:2189-2198, incorporated herein by reference).

Because the object of the test is to find very small polyps, which when removed will never become cancerous, it is necessary be sure the colon is free of all residue and fecal material so that there is no confusion in the results of the test. A critical factor for the success of the VC screening trial reported in the New England Journal of Medicine (supra) was the particular method of colon preparation used for the VC examination, whereas the poor results of the previous VC trials were due, at least in part, to the inadequacies of their colon preparation.

There are three key issues related to the VC colon preparation that must be addressed:
1) clearing the colon of as much fecal material as possible (cathartic cleansing);
2) finding a way to tag any residual fecal material left behind so that it can be distinguished from true colonic polyps; and
3) finding a way to tag or opacify any remaining colonic fluid so that submerged polyps can be detected.
   Previous VC colon preparations have largely focused on only one of these issues, for example: cathartic cleansing (See: Fenlon H M, et al. A Comparison of Virtual and Conventional Colonoscopy for the Detection of Colorectal Polyps. N Engl J Med 1999; 341:1540-1542; and Yee J., et al. Colorectal Neoplasia: Performance Characteristics of CT Colonography for Detection in 300 Patients. Radiology 2001; 219:685-692). A great need has existed for an improved preparation that addresses all three if the issues outlined above.

Cathartic cleansing of the colon is required for effective colonoscopy, whether it is performed by conventional or virtual methods. Incomplete cleansing of the colon prior to VC examination decreases accuracy in two ways: 1) decreased sensitivity due to obscuration of colonic polyps by residual fecal material, and 2) decreased specificity because residual stool is the major reason for a false positive on VC examinations (See: Pickhardt P. J., et al. Electronic Cleansing and Stool Tagging in CT Colonography: Advantages and Pitfalls Encountered with Primary Three-Dimensional Evaluation. AJR 2003; 181:799-805, incorporated herein by reference; and Yee J., CT Colonography: Examination Prerequisites. Abdom Imaging 2002; 27:244-252).

Commonly used agents for cleansing prior to CC include polyethylene glycol (PEG) and sodium phosphate (NaP). PEG is an electrolyte lavage preparation that requires the patient to drink large volumes of the solution. NaP is a saline cathartic that produces an osmotic load by drawing fluid into the colon. Numerous published studies have compared PEG and NaP for colonic cleansing before CC examination, including a meta-analysis of all these trials, which concluded that the oral NaP was better tolerated and more efficacious (See: Hsu C. W., et al. Meta-Analysis and Cost Comparison of Polyethylene Glycol Lavage Versus Sodium Phosphate for Colonoscopy Preparation. Gastrointest Endosc 1998; 48:276-282; Cohen S. M., et al. Prospective, Randomized, Endoscopic-Blinded Trial Comparing Precolonoscopy Bowel Cleansing Methods. Dis Colon Rectum 1994; 37:689-696; and Vanner S. J., et al. A Randomized Prospective Trial Comparing Oral Sodium Phosphate with Standard Polyethylene Glycol-Based Lavage Solution (Golytely) in the Preparation of Patients for Colonoscopy. Am J Gastroenterol 1990; 85; 422-427). By direct extension, NaP is also the preferred preparation for cleansing prior to VC examination. Furthermore, it has been shown that NaP results in significantly less residual colonic fluid at VC compared with PEG (See: Macari M., et al. Effect of Different Bowel Preparations on Residual Fluid at CT Colonography. Radiology 2001; 218:274-277), which is also highly advantageous. For patients with known chronic renal failure or congestive heart failure, magnesium citrate, another saline cathartic, can be substituted for NaP. Bisacodyl tablets, with or without rectal suppository, are often given in conjunction with the cathartic agent to supplement cleansing.

Residual fecal material is the major cause of false positive results at VC examination since it can mimic the appearance of colonic polyps. In the setting of VC for primary polyp screening, this could lead to unnecessary referrals for CC. Therefore, accurate performance of VC requires that the radiologist can distinguish residual stool from true colonic polyps. Marking or “tagging” residual fecal material with oral barium sulfate, which is given as part of the colon prep, has been shown to be effective for making this distinction (See: Pickhardt P. J., et al. AJR 2003; 181:799-805, Supra), which was subsequently proven in the screening trial (See: Pickhardt P. J., et al. N Engl J Med 2003; 349:2189-2198, Supra). Barium fills the interstices of residual stool, which causes increased internal density that is readily detectable by CT VC, whereas true colonic polyps do not take up barium internally. This orally administered barium does not interfere with subsequent CC examination (See: Pickhardt P. J., Virtual Colonoscopy to Screen for Colorectal Cancer (reply). N Engl J Med 2004; 350: 1148-1150, incorporated herein by reference), in the event it is needed. Some investigators are using barium sulfate without a cathartic to study a so-called “prepless” approach to VC (See: Callstrom M. R., et al. CT Colonography Without Cathartic Preparation: Feasibility Study. Radiology 2001; 219:693-698; and Lefere P. A., Dietary Fecal Tagging as a Cleansing Method Before CT Colonography: Initial Results—Polyp Detection and Patient Acceptance. Radiology; 2002; 224:393-403). This method, however, has not yet been proven in clinical screening for colonic polyps. Furthermore, if a significant polyp is found, the patient would still need to undergo cathartic cleansing prior to CC.

Barium sulfate does not adequately opacify the residual colonic fluid and, therefore, does not address the third key issue. Polyps submerged in the residual colonic fluid will often be missed at VC because the polyp and fluid are of similar density on a CT scan without intravenous contrast. Orally administered water soluble iodinated contrast agents, such as iohexol (omnipaque) or diatrizoate meglumine/diatrizoate sodium (gastrografin or gastroview), can be used as part of a VC prep to uniformly opacify the residual colonic fluid that remains after cleansing (See: Pickhardt P. J., et al. AJR 2003; 181:799-805, Supra; and Miller M. T., Assessment of Bowel Opacification on Oral Contrast-Enhanced CT Colonography: Multi-Institutional Trial. 2004 annual meeting for the Society of Gastrointestinal Radiologists). The resulting increased density of the fluid allows for the detection of polyps at VC that are submerged in this fluid. Fluid opacification also allows for its “virtual” removal from the CT images by a process called “electronic fluid cleansing” or “digital subtraction” (See: Pickhardt P. J., et al. AJR 2003; 181:799-8058 Supra; and Zalis M. E., CT Colonography: Digital Subtraction Bowel Cleansing with Mucosal Reconstruction: Initial Observations. Radiology 2003; 226:911-917). Unfortunately, water-soluble contrast agents do not adequately tag residual solid stool, so barium sulfate is still needed to address this issue.

As outlined above, the prior art of VC colonic preparation was at a nascent stage of development, which is not surprising as the concept of VC itself is only 10 years old. Current VC colonic preparations have typically addressed only one of the three key issues noted above. Most attempts directly applied previous CC preps by incorporating only cathartic cleansing (See Johnson C. D., et al. Gastroenterology 2003; 125:311-3193 Supra; Fenlon H M, et al. N Engl J Med 1999; 341:1540-1542 Supra; and Yee J., et al. Radiology 2001; 219:685-692 Supra), which fails to deal with the problems of residual colonic stool or fluid. Other efforts have dealt with only tagging of solid stool (See: Callstrom M. R., et al. Radiology 2001; 219:693-698 Supra; and Lefere P. A., Radiology; 2002; 224:393-403 Supra), the efficacy of which has yet to be studied in a large patient population. One previous effort incorporated cathartic cleansing with water-soluble contrast opacification of fluid (See: Pineau B. C., et al. Virtual Colonoscopy Using Oral Contrast Compared with Colonoscopy for the Detection of Patients with Colorectal Polyps. Gastroenterology 2003; 125:304-310 Supra), but did not tag solid residual stool and also did not employ electronic subtraction to virtually remove fluid.

### BRIEF SUMMARY OF THE INVENTION

The present invention presents an oral colonic preparation for VC examination that combines a cathartic for cleansing, barium sulfate for stool tagging, and water-soluble iodinated contrast for fluid opacification. Unlike the prior art, this unique combination addresses all three problems that must be handled for effective colonic preparation prior to VC examination. Furthermore, the efficacy of this combination has been proven in a large multi-center VC screening trial (See: Pickhardt P. J., et al. N Engl J Med 2003; 349:2189-2198, Supra), which is the largest VC study to date and the first to evaluate a true screening population.

In one preferred embodiment, effective split doses of sodium phosphate (NaP), barium sulfate, and diatrizoate meglumine/diatrizoate sodium are all taken orally beginning the day prior to the VC examination. Oral bisacodyl tablets are also included in this preferred embodiment to further supplement cleansing. For patients with a relative contraindication to NaP, other preferred embodiments include oral magnesium citrate or polyethylene glycol (PEG) in place of NaP for cleansing, along with the barium sulfate, water soluble iodinated contrast, and bisacodyl tablets and/or suppositories.

### DETAILED DESCRIPTION OF THE INVENTION

The instant invention combines three distinct components to form a single effective bowel preparation for VC examination:
1) a cathartic for colonic cleansing;
2) barium sulfate for stool tagging; and
3) water soluble iodinated contrast for colonic fluid opacification.

Although each separate component has been individually employed previously for VC preparation, the prior art has experienced sub-optimal results because all three components together have never been combined. The unique bowel preparation described herein combines these components and has been proven highly effective in a large, prospective multi-center VC screening trial (See: Pickhardt P. J., et al. N Engl J Med 2003; 349:2189-2198, Supra), the first of its kind. In addition to proving its efficacy, the packaging of these three components into a single preparation kit would be much more convenient for patients and their referring physicians.

The preferred embodiments of this invention are outlined below. The specific timing and dosage of each component can vary within an acceptable effective range. In general, the preparation begins the day before the patient is scheduled for VC examination and also and includes a clear liquid diet.

Cathartic cleansing of the colon is essential for effective VC examination. The amount of residual stool and fluid needs to be limited as much as reasonably possible since both will negatively impact VC accuracy for detecting colonic polyps. Previously known colonic preparations for CC examination have shown that oral sodium phosphate (NaP), a saline cathartic, is preferable to polyethylene glycol (PEG), an electrolyte lavage, since NaP is much better tolerated by patients and is more effective (Hsu C. W., et al. Gastrointest Endosc 1998; 48:276-282, Supra; Cohen S. M., et al. Dis Colon Rectum 1994; 37:689-696, Supra; and Vanner S. J., et al. Am J Gastroenterol 1990; 85; 422-427, Supra). For these reasons and because NaP results in significantly less residual colonic fluid compared with PEG (See: Macari M., et al. Radiology 2001; 218:274-277, Supra), oral NaP is also the preferred cleansing agent for VC. The effective dose range for oral NaP (generally, 15-45 g/50 ml, mono- and di-sodium phosphate) prior to VC examination is 20-90 ml, split into individual doses of up to 50 ml each. Bisacodyl tablets (5-20 mg) are included in a preferred embodiment to supplement cleansing. The preferred embodiment for patients with known chronic renal failure or congestive heart failure consists of magnesium citrate (200-600 ml), another saline cathartic, in place of NaP. Bisacodyl tablets (5-20 mg) and suppository (5-20 mg) supplement cleansing in this embodiment. Another preferred embodiment for patients with known chronic renal failure or congestive heart failure consists of PEG (4 L) in place of NaP.

Residual fecal material is the major cause of false positive results at VC examination, since it can mimic the appearance of colonic polyps. Tagging residual fecal material with oral barium sulfate, taken the day before VC examination, is effective for making this distinction (See: Pickhardt P. J., et al. AJR 2003; 181:799-805, Supra). In a preferred embodiment, 100-600 ml of dilute barium sulfate suspension (1.5-2.5 w/w) is used, which can be divided into two individual doses of 30-300 ml each. In another preferred embodiment, barium concentrations of up to 40% may be used to decrease the overall volume, as desired. Barium fills the interstices of residual stool and creates high internal density within the lesions that is radiopaque and readily distinguishable from true colonic polyps at VC examination (See: Pickhardt P. J., et al. AJR 2003; 181:799-805, Supra). This orally administered barium does not interfere with subsequent CC examination, in the event it is needed (See: Pickhardt P. J., N Engl J Med 2004; 350: 1148-1150, Supra). Barium sulfate, however, does not adequately opacify residual colonic fluid and therefore cannot be utilized for effective fluid tagging. Opacification of residual colonic fluid with a radiopaque substance is important for the detection of submerged colonic polyps at VC examination. Without fluid opacification, submerged polyps will generally be missed because the polyp and fluid are of similar density on a CT scan without intravenous contrast. In a preferred embodiment, a water-soluble iodinated contrast agent such as iohexol (i.e. 50-75 ml, 300-350 mg I/ml) or diatrizoate meglumine/diatrozate sodium (gastrografin/gastroview, 30-50% bound iodine, 0.40-80 ml), is orally ingested to uniformly opacify any residual colonic fluid. The previous doses can be delivered/taken all at once or can be divided. In another embodiment, more concentrated iodinated agents, such as iohexol, can be used to decrease the overall volume the patient must drink. The resulting increased density of the fluid allows for the detection of polyps at VC that are submerged in this fluid. Fluid opacification also allows for the “virtual” removal of this fluid from the CT images by a process called “electronic fluid cleansing” or “digital subtraction” (See: Pickhardt P. J., et al. AJR 2003; 181:799-805, Supra; and Zalis M. E., Radiology 2003; 226:911-917, Supra). Iodinated water-soluble contrast agents do not adequately tag residual solid stool, so barium sulfate is still needed to address this issue.

Example 1 provides a summary of one preferred embodiment. Example 2 details some of the acceptable variations or substitutions for the elements outlined in Example 1. A sample regimen for a preferred embodiment of the VC colon preparation is outlined in Example 3. Example 4 contains a generalized treatment regimen for the Virtual Colonoscopy Preparation. Specific timing and dosage can both vary within a reasonable range, since the key is really the actual combination of a cathartic with barium sulfate and iodinated contrast. The specific choice for a clear supplemental beverage can of course be varied considerably. Likewise, the available options for a clear liquid diet are widely variable. As noted above, other preferred embodiments may include magnesium citrate or PEG, particularly in patients with chronic renal failure or congestive heart failure. Also, for lower volume preps, barium sulfate concentration can be increased to 40%, and more concentrated iodinated contrast agents can also be employed. Bisacodyl rectal suppository is also optional in some preferred embodiments.

### EXAMPLE 1

### Summary of a Preferred Embodiment

1. Oral cathartic—NaP (sodium phosphate, phospho-soda)
2. Oral barium—barium sulfate 2% w/w barium
3. Oral iodinated contrast—iohexol (300-350 mg I/ml)

### EXAMPLE 2

### Acceptable Substitutions/Variations

1. Oral cathartic:
   Magnesium citrate
   PEG
2. Oral barium: any barium salt, w/w up to 40%
3. Oral iodine contrast:
   gastrografin/gastroview (diatrizoate meglumine/diatrizoate sodium)
   Other iodinated contrast agents

Concentrations and volumes of above components can be varied within an acceptable range. Bisacodyl tablets may be added to any of the above combinations

### EXAMPLE 3

### Sample Patient Instructions for Virtual Colonoscopy Prep

On the Day Before the VC Exam

All Day: Follow a restricted diet consisting of clear liquids (only clear soup, juice, carbonated drinks, and water). Drink plenty of fluid throughout the day to avoid dehydration. Ensure that you have easy access to a restroom.

| Time | Instruction |
|---|---|
| 12:00 PM | Take two (2) bisacodyl tablets with 125-300 ml of 1.5-2.1% |
| | barium sulfate |
| 3:00 PM | Drink 45 ml of sodium phosphate prep with 8 ounces of clear |
| | juice or clear carbonated drink. |
| | Follow this with 4-8 cups of water throughout the |
| | afternoon |
| 6:00 PM | Drink 20-45 ml of sodium phosphate prep with 8 ounces of |
| | clear juice or clear carbonated drink. |
| | Follow this with 4 to 8 cups of water throughout the evening. |
| 9:00 PM | Drink ½ of chilled bottle (60 ml) of diatrizoate meglumine/diatrizoate |
| | sodium mixed with Sprite or clear juice and 125-300 ml of |
| | 1.5-2.1% barium. |

On the Day Before the VC Exam

| | |
|---|---|
| Time | Instruction |

| | |
|---|---|
| 2 hours | Drink ½ of chilled bottled (60 cc) of diatrizoate |
| before | meglumine/diatrizoate sodium with 8 oz of clear juice or |
| study | Sprite and drink 1 ½ to 2 hours before virtual colonoscopy. |

### EXAMPLE 4

### Generalized Treatment Regimen for Virtual Colonoscopy Prep

In order to prepare a patient's colon for a colonoscopy, the patient takes a treatment regimen of an oral cathartic cleansing solution, an oral barium solution, and an oral iodinated contrast solution. The active ingredient for the cathartic cleansing solution is sodium phosphate, magnesium citrate, polyethylene glycol, or combinations thereof.

When the sodium phosphate cathartic cleansing solution is used, it can be delivered orally to the patient in one or more doses of 20 to 50 ml. The active ingredient for the barium solution is barium sulfate (1.0 to 40 percent w/w, delivered orally in one or more doses of 30 to 300 ml). The active ingredient for the iodinated contrast solution is diatrizoate meglumine, diatrizoate sodium, iohexol, other iodinated contrast agents, or any combination thereof. The treatment regimen can further include the administering 5 to 20 mg of bisacodyl, either orally or as a suppository.

We claim:

1. A method of preparing a patient's colon for a colonoscopy, comprising administering to said patient a treatment regimen of an oral cathartic cleansing solution, an oral barium solution, and an oral iodinated contrast solution.

2. The method of claim 1, wherein the active ingredient for said cathartic cleansing solution is selected from the group consisting of sodium phosphate, magnesium citrate, polyethylene glycol, and combinations thereof.

3. The method of claim 1, wherein the active ingredient for said cathartic cleansing solution is sodium phosphate.

4. The method of claim 3, wherein the sodium phosphate cathartic cleansing solution is delivered orally to said patient in at least one dose of 20 to 50 ml.

5. The method of claim 1, wherein the active ingredient for said barium solution is barium sulfate.

6. The method of claim 5, wherein concentration of said barium sulfate is 1.0 to 40 percent w/w of said solution.

7. The method of claim 5, wherein concentration of said barium sulfate is 1.5 to 2.5 percent w/w of said solution.

8. The method of claim 7, wherein said barium sulfate solution is delivered orally to said patient in at least one dose of 30 to 300 ml.

9. The method of claim 1, wherein the active ingredient for said iodinated contrast solution is selected from the group consisting of diatrizoate meglumine, diatrizoate sodium, iohexol, other iodinated contrast agents, and combinations thereof.

10. The method of claim 1, wherein said treatment regimen further comprises the oral administering to said patient of at least one dose of 5 to 20 mg of bisacodyl.

11. The method of claim 1, wherein said treatment regimen further comprises the suppositoral delivery to said patient of at least one dose of 5 to 20 mg of bisacodyl.
